(19) United States
(12) Patent Application Publication
(10) Pub. No.: US 2026/0118343 A1
(43) Pub. Date: Apr. 30, 2026

(54) METHODS AND APPARATUS FOR HEMOLYSIS DETECTION

(71) Applicant: Siemens Healthcare Diagnostics Inc., Tarrytown, NY (US)

(72) Inventor: Jeffery Franklin, Nepean (CA)

(73) Assignee: Siemens Healthcare Diagnostics Inc., Tarrytown, NY (US)

(21) Appl. No.: 19/149,246

(22) PCT Filed: Jan. 25, 2024
(86) PCT No.: PCT/US2024/012932

§ 371 Date: Jul. 18, 2025

Related U.S. Application Data
(60) Provisional application No. 63/482,123, filed on Jan. 30, 2023.

(51) Int. Cl. G01N 33/49

(52) U.S. Cl. CPC G01N 33/4925

(57) ABSTRACT

A sensor assembly enabling hemolysis detection with a diagnostic analyzer. The sensor assembly can be included in a diagnostic cartridge that may be coupled to the diagnostic analyzer and a sample fluid can be delivered thereto. The sensor assembly includes a main oxygen sensor configured to provide a first measurement and a modified oxygen sensor including an oxidant configured to oxidize hemoglobin iron to methemoglobin and configured to provide a second measurement. A hemolysis detection module of the diagnostic analyzer provides a level of hemolysis in the sample based on a difference between the first measurement and the second measurement. Numerous other diagnostic analyzers, diagnostic cartridges, and detection methods are provided.

This application claims benefit under 35 USC § 119 (e) of U.S. Provisional Application No. 63/482,123, filed Jan. 30, 2023. The entire contents of the above-referenced patent application are hereby expressly incorporated herein by reference.

### FIELD

The present application relates to diagnostic testing methods and apparatus, and more particularly to methods and apparatus enabling a detection of a level of hemolysis in a blood sample.

### BACKGROUND

Hemolysis is the destruction of red blood cells, which, in some instances, can result from phlebotomy or pre-analytical causes associated with sample handling. For example, hemolysis of a blood sample may be caused by using an incorrect needle size, improper tube mixing, incorrect filling of tubes, excessive suction usage, prolonged tourniquet application, prolonged storage, extreme temperature, delayed processing, and/or other difficulties in the collection of a blood sample or in sample handling.

Similarly, hemolysis in a blood sample can be present due to certain illnesses or medical conditions, such as hemolytic anemia, autoimmune conditions, bone marrow failure, and inherited blood conditions such as sickle cell disease or thalassemia.

Hemolysis has traditionally been detected by a manual visual inspection of the blood sample by a technician after separation of the plasma (or serum) portion (i.e., through centrifugation) and comparing the plasma (or serum) color with a colored hemolytic chart. The chart shows colors of separated samples associated with increasing concentration of free (extracellular) hemoglobin contained in the plasma (or serum). Thus, the technician can ascribe a hemolytic index based on the visual color of the separated plasma (or serum).

Hemolysis due to improper or mishandled procedures during specimen collection is the most undesirable precondition that can influence accuracy of the results and dependability of blood gas testing. The impact of in vitro hemolysis on measured potassium concentrations, for example, is well known. In such cases, reported potassium concentrations can be clinically inaccurate, at a magnitude dependent on the degree of hemolysis. Many other analytes can be impacted by the biological and analytical interference effects of in vitro hemolysis. For example, when free (extracellular) hemoglobin is present in a blood sample, it can have properties that can cause interference in certain types of diagnostic testing, such as diagnostic assays that include measurements by an optical measurement technique. Particularly, free extracellular hemoglobin present in the sample can interfere with certain assays due to its absorption properties. Thus, identifying samples containing hemolysis is desirable for laboratory or point of care testing so that certain results can be flagged as possibly suspect and/or redrawn. Furthermore, automated testing is desirable to minimize the subjective nature of manual visual hemolysis determination made by medical personnel, but also to speed the hemolysis detection process. Elimination of the process of centrifugation in order to test for hemolysis would also be beneficial.

Thus, apparatus and methods enabling automated hemolysis detection and improved speed and that can minimize or eliminate the judgement and inconsistencies associated with manual visual inspection are desired.

### SUMMARY

In some embodiments provided herein, a diagnostic sensor assembly is provided. The diagnostic sensor assembly may be embodied in a diagnostic cartridge that is configured for connection to a diagnostic analyzer in order to determine a level of hemolysis in a sample. The diagnostic sensor assembly comprises a sample inlet configured to receive a sample, a sample passageway extending from the sample inlet, a main oxygen sensor configured to contact the sample along the sample passageway, and a modified oxygen sensor configured to contact the sample along the sample passageway, the modified oxygen sensor comprising an oxidant configured to oxidize hemoglobin to methemoglobin (Fe⁻ to Fe³⁺), i.e., changing its heme iron configuration from the ferrous state to the ferric state. From signals obtained from the main oxygen sensor and the modified oxygen sensor, quantification of a level of hemolysis in the sample can be obtained.

In some embodiments provided herein, a diagnostic analyzer comprises an analyzer body including a cartridge receiver and an electrical connector, a diagnostic cartridge receivable in the cartridge receiver, the diagnostic cartridge comprising: a cartridge body configured to be coupled to the cartridge receiver, the cartridge body including: electrical contacts couplable to the electrical connector when the cartridge body is received into the cartridge receiver, a sample inlet configured to receive a sample, a sample passageway extending into the cartridge body from the sample inlet and configured to receive the sample therein, a main oxygen sensor configured to contact the sample in the sample passageway and configured to provide a first measurement of the sample, a modified oxygen sensor configured to contact the sample in the sample passageway, the modified oxygen sensor including an oxidant configured to oxidize hemoglobin to methemoglobin (Fe²⁺ to Fe³⁺) and configured to provide a second measurement of the sample, and a controller coupled to the electrical connector, the controller configured to receive the first measurement and the second measurement of the sample and configured to provide a level of hemolysis in the sample based on the first measurement and the second measurement.

In some embodiments provided herein, a method of determining hemolysis of a sample is provided. The method comprises coupling a diagnostic cartridge to a cartridge receiver of a diagnostic analyzer, the diagnostic cartridge comprising a sample inlet configured to receive a sample, a sample passageway extending into the cartridge body from the sample inlet, a main oxygen sensor configured to contact the sample in the sample passageway, a modified oxygen sensor configured to contact the sample in the sample passageway, the modified oxygen sensor including an oxidant configured to oxidize hemoglobin to methemoglobin (Fe²⁺ to Fe³⁺); passing the sample through the sample passageway and into contact with the main oxygen sensor and the modified oxygen sensor; obtaining a first measurement of the sample from the main oxygen sensor; obtaining a second measurement of the sample from the modified oxygen sensor; and providing a level of hemolysis in the sample based on the first measurement and the second measurement.

Other features and aspects of the present disclosure will become more fully apparent from the following detailed description, claims, and the accompanying drawings.

### BRIEF DESCRIPTION OF THE DRAWING

The drawings, described below, are for illustrative purposes only, and are not necessarily drawn to scale. The drawings are not intended to limit the scope of the disclosure in any way.

FIG. 1 illustrates a perspective view of an example of a diagnostic analyzer (e.g., for a point-of-care location) including a diagnostic cartridge (comprising a diagnostic sensor assembly) that is receivable in a cartridge receiver accordance with embodiments provided herein.

FIG. 2 illustrates a bottom plan view of an example of a diagnostic cartridge containing a sensor assembly configured to provide measurements used in hemolysis level detection in accordance with embodiments provided herein.

FIG. 3A illustrates a partial cross-sectioned view of an example of the diagnostic cartridge taken along section lines 3A-3A of FIG. 2 and illustrating an example construction of the sensor assembly wherein the oxidant is included in an outer membrane layer covering an internal electrolyte chamber. That membrane layer may be homogeneous or heterogenous in accordance with embodiments provided herein.

FIG. 3B illustrates a partial cross-sectioned view of another example of the diagnostic cartridge like FIG. 3A with the oxidant compounded into a membrane layer. That membrane layer may be homogeneous or heterogeneous in accordance with embodiments provided herein.

FIG. 3C illustrates a partial cross-sectioned view of an example of the modified oxygen sensor with the oxidant compounded into the membrane in accordance with embodiments provided herein.

FIG. 3D illustrates a schematic top view of an example of a diagnostic cartridge including the main and modified oxygen sensors as well as a reference sensor and other additional sensors and common ground in accordance with embodiments provided herein.

FIG. 3E illustrates a partial cross-sectioned view of an example of the modified oxygen sensor with the oxidant compounded into an outermost layer of a multi-layer membrane in accordance with embodiments provided herein.

FIG. 4 illustrates a schematic diagram of an example of a diagnostic analyzer including a diagnostic cartridge containing a sensor assembly receivable therein and illustrating the sensor components thereof and their connection to a controller of the diagnostic analyzer in accordance with embodiments provided herein.

FIG. 5 illustrates a bottom plan view of another example of a diagnostic cartridge containing a sensor assembly configured to provide measurements used in hemolysis level detection wherein the sample passageway thereof includes multiple forks or legs that may split off from a main passageway in accordance with embodiments provided herein.

FIG. 6 illustrates a cross-sectional view of a first passageway portion that may split off from a main passageway of the example diagnostic cartridge taken along section lines 6-6 of FIG. 5 in accordance with embodiments provided herein.

FIG. 7 illustrates a cross-sectional view of a second passageway portion that may split off from the main passageway of the example diagnostic cartridge taken along section 7-7 of FIG. 5 in accordance with embodiments provided herein.

FIG. 8 illustrates a flowchart of an example method of detecting hemolysis using a diagnostic sensor assembly and diagnostic analyzer in accordance with embodiments of the present disclosure.

### DETAILED DESCRIPTION

Independent of the grammatical term usage, individuals with male, female or other gender identities are included within the term.

In some embodiments, a diagnostic sensor assembly is provided. The diagnostic sensor assembly may be embodied in a diagnostic cartridge (e.g., a card like member) that includes a cartridge body containing a passageway and multiple sensors therein. The diagnostic cartridge may be, for example, a single-use cartridge in some embodiments. Diagnostic cartridge may be received by (e.g., inserted into or otherwise coupled to) a diagnostic analyzer that is configured to provide blood analysis, and, in particular, a hemolysis level detection (quantification) of a blood sample. The diagnostic analyzer may be, for example, a handheld or a benchtop diagnostic analyzer. The blood sample may be provided to the diagnostic sensor assembly of the diagnostic cartridge. The blood sample may be whole blood, for example. Optionally, the sample may be blood serum or plasma, which may contain hemolysis. The volume of whole blood used by the test may be very small, such as 100 μL or less.

When the sensor assembly is included in a diagnostic cartridge, the cartridge body that is configured for connection with a diagnostic analyzer can be provided in any suitable configuration in order to provide measurement signals from the various sensors of the diagnostic cartridge. The cartridge body can include the diagnostic sensor assembly therein having a sample inlet configured to receive a sample therein, and a sample passageway extending from the sample inlet, such as into the cartridge body. Sample (e.g., whole blood) may be provided (e.g., injected) into the sample passageway from the sample inlet, such as by use of a syringe, a pumping mechanism, or other suitable sample transfer device.

The diagnostic sensor assembly of diagnostic cartridge further comprises multiple sensors therein. In the described embodiments herein, the sensor assembly comprises two oxygen sensors. In particular, the sensor assembly comprises a main oxygen sensor that is configured to contact the sample along the sample passageway, and a modified oxygen sensor that is also configured to contact the sample along the sample passageway. The modified oxygen sensor is different from the main oxygen sensor in that the modified oxygen sensor comprises (i.e., includes or is associated with) an oxidant, where the oxidant is configured to oxidize extracellular hemoglobin in the sample to methemoglobin (Fe²⁺ to Fe³⁺). In particular, in some embodiments, the modified oxygen sensor comprises an oxidant such that can be provided in a membrane of the modified oxygen sensor. In other embodiments, the modified oxygen sensor comprises an oxidant such that the oxidant is positioned not in the membrane, but in the sample passageway proximate to (e.g., upstream of) an oxygen sensor enabling the sample to flow over the oxidant prior to flowing over the oxygen sensor. In this manner, the extracellular hemoglobin is oxidized prior to reaching the proximate oxygen sensor. Thus, this embodiment involves measuring a “modified” level of oxygen in the sample, hence the term modified oxygen sensor also referring to the combination of the upstream oxidant and the proximate oxygen sensor. The amount of sample modification would be dependent on the degree and presence of hemolysis within the sample owing to the upstream reaction of the oxidant provided in the passageway with the sample.

In each of the embodiments, quantification of a level of hemolysis of the sample can be obtained based on the signals generated by the main oxygen sensor and the modified oxygen sensor. The signals are indicative of the respective localized O₂ measurements proximate the main and modified sensors of the diagnostic sensor assembly. These signals may be provided to a controller of the diagnostic analyzer for processing and to make a hemolysis level determination.

Due to the presence of the oxidant, the oxidation occurring proximate the modified oxygen sensor liberates oxygen if there is free (extracellular) hemoglobin present in the sample and thus achieves a higher level of localized oxygen for the modified oxygen sensor to sense as compared to the main oxygen sensor that does not include the oxidant. The oxidant is configured to be in contact with the sample. A difference between the oxygen signals of the main oxygen sensor and the modified oxygen sensor can be correlated to a level of hemoglobin present in the sample and thus can provide detection (quantification) of a level of hemolysis in the sample. The level of hemolysis can be processed to provide a hemolytic index. The index can range from zero to a maximum value, for example, and can be displayed to the operator of the diagnostic analyzer or otherwise communicated electronically within a hospital information system. A calibration may be accomplished before (or even after) running the hemolysis detection to ensure that the diagnostic sensor assembly will produce proper results.

In another embodiment, a diagnostic analyzer configured to provide detection (quantification) of hemolysis in a blood sample is provided. The diagnostic analyzer has an analyzer body including a cartridge receiver and an electrical connector. The electrical connector is configured to make an electrical connection between a controller and the diagnostic sensor assembly configured in the diagnostic cartridge receivable in the cartridge receiver. The cartridge body is configured to be received by the cartridge receiver, where the cartridge body includes electrical contacts that are configured to couple to the electrical connector when the cartridge body is received by the cartridge receiver. The diagnostic sensor assembly of the cartridge body including the sample inlet and sample passageway is configured to receive the sample therein.

The main oxygen sensor is configured to come into contact with the sample provided in the sample passageway and configured to provide a first measurement (a signal correlated to an amount of oxygen proximate to the main oxygen sensor) of the sample. Similarly, the modified oxygen sensor is configured to come into contact with the sample provided in the sample passageway and the oxidant (of the modified oxygen sensor) is operable to oxidize extracellular hemoglobin to methemoglobin (change from Fe²⁺ to Fe³⁺). Thus, the modified oxygen sensor is configured to provide a second measurement (a signal correlated to an amount of oxygen in the sample proximate to the modified oxygen sensor).

The controller of the diagnostic analyzer is electrically coupled to the electrical connector (of the analyzer body). The electrical connector may include a plurality of conductive paths in order to connect to the plurality of electrical contacts (of the sensor assembly) and convey the sensor information from each of the sensors of the diagnostic sensor assembly. In particular, controller is configured to receive, through the connection between the electrical connector and the electrical contacts of the diagnostic sensor assembly, the first measurement and the second measurement.

The controller is further configured to provide a hemolysis level measurement (quantification) based upon the first measurement and the second measurement. In particular, the processing is undertaken by a processor of the controller wherein a hemolysis detection module thereof is configured to execute a difference-finding routine. The difference between the first measurement and the second measurement, obtained by subtraction, is correlated to a degree of hemolysis present in the sample. In particular, an elevated second measurement as compared to the first measurement is quantitatively correlated to a degree (index level) of hemolysis in the sample, where a higher index level indicates a larger quantity of hemolysis in the sample.

In yet another embodiment, a method of detecting hemolysis of a sample (e.g., whole blood, plasma, or serum) is provided. The method comprises coupling a diagnostic sensor assembly to a diagnostic analyzer. The sensor assembly may be embodied as part of a diagnostic cartridge that is couplable, i.e., configured to couple to, a cartridge receiver of the diagnostic analyzer. The sensor assembly of the diagnostic cartridge can comprise a sample inlet configured to receive a sample, a sample passageway extending from the sample inlet (e.g., such as into the cartridge body), a main oxygen sensor configured to contact the sample in the sample passageway, and a modified oxygen sensor configured to contact the sample in the sample passageway, where the modified oxygen sensor comprises an oxidant configured to oxidize extracellular hemoglobin in the sample to methemoglobin (Fe²⁺ to Fe³⁺). The method further comprises passing the sample through the sample passageway and into contact with the main oxygen sensor and the modified oxygen sensor, and obtaining a first measurement of the sample from the main oxygen sensor and obtaining a second measurement of the sample from the modified oxygen sensor. The measurements may comprise signals correlated with localized oxygen readings. The first measurement is correlated to a first partial pressure measurement of oxygen in the sample proximate the main oxygen sensor. The second measurement is correlated to a second partial pressure measurement of oxygen in the sample proximate the modified oxygen sensor. According to the method, a level of hemolysis is determined based on the first measurement and the second measurement that quantifies a degree (index level) of hemolysis contained in the sample.

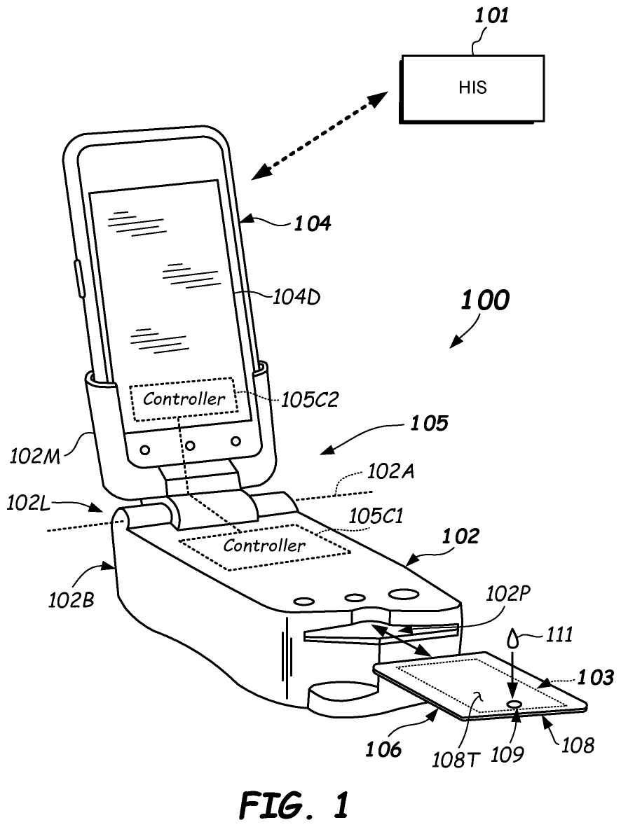

These and other embodiments of the present disclosure are fully described herein with reference to FIGS. 1-8 herein.

With reference to FIG. 1, a perspective view of an example of a diagnostic analyzer 100 is shown in accordance with embodiments provided herein. Diagnostic analyzer 100 includes an analyzer body 102 configured to house various user interfaces such as user control haptics (e.g., buttons, switches, touch screens, and the like). In the depicted embodiment, the analyzer body 102 of the diagnostic analyzer 100 can comprise a computing device 104. Computing device 104 can be detachably mounted to a device mount 102M of a base 102B of the diagnostic analyzer 100 in some embodiments. Computing device 104 can be a hand-held computing device such as a personal digital assistant (PDA), tablet, or other like computing device. In some diagnostic analyzers, the processing and memory functions of the computing device 104 may be housed inside of the analyzer body 102 rather than as a separable/detachable version of the computing device 104.

The diagnostic analyzer 100 includes a controller 105, which in this embodiment is configured to include a first controller 105C1, which can be part of the base 102B of the analyzer body 102, and a second controller 105C2, which can be part of, or integral with, the computing device 104. The first controller 105C1 and the second controller 105C2 are in electronic communication with one another and can perform different functions. In this embodiment, the computing device 104 can include a display 104D enabling user input, visual display of operational information, test results, and other information. In some embodiments, the display 104D may be tiltable about a pivot axis 102A. For example, the device mount 102M of the base 102B may receive the computing device 104 and can be pivotable about the pivot axis 102A at a location 102L so as to allow adjustment of the viewing angle.

The display 104D can be a touch screen having a user interface that allows an operator, in conjunction with one or more haptics (e.g., button, switches, or other user controlled devices), to control operation of the diagnostic analyzer 100, observe measurement results from diagnostic testing performed, and/or other ancillary functions. The first controller 105C1 may include electronics enabling communication with the diagnostic sensor assembly 103 embodied in the diagnostic cartridge 106 including signal conditioning (including filtering, A/D conversion, and/or possibly amplification) of the various sensor signals received from various sensors of the diagnostic sensor assembly 103 of the diagnostic cartridge 106 to be described in more detail herein. The first controller 105C1 may further include electronics enabling the provision of amperometric or potentiometric inputs to the diagnostic sensor assembly 103 as a baseline input for carrying signals correlated to the sensor measurement signals.

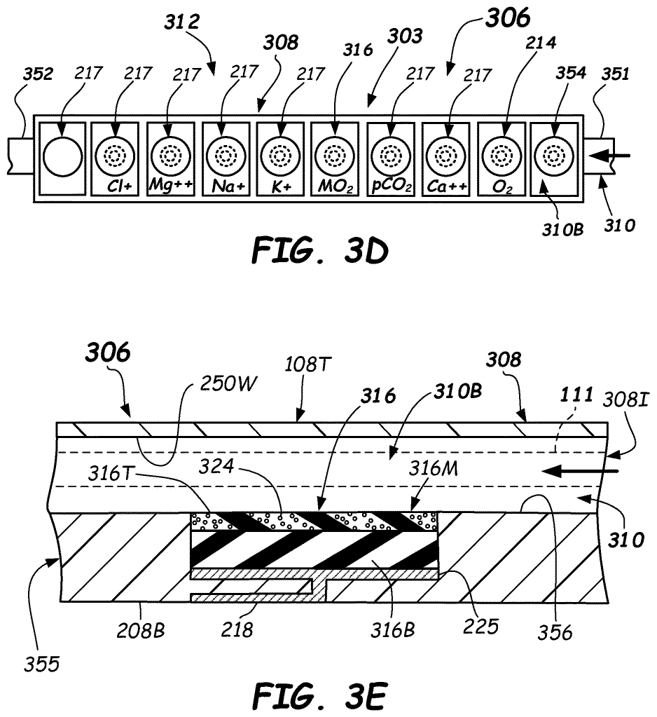

The second controller 105C2 can be operable to perform processing and can include a hemolysis detection module 430 (FIG. 4) that is a software module containing a difference finding routine. The software module containing the difference finding routine may be stored in a memory 431 of the diagnostic analyzer 100 and may be executable on a processor 432. The hemolysis detection module 430 receives sensor readings (signals) from the various oxygen sensors to be described herein and compares their values to obtain a difference there between. From this difference, the hemolysis detection module 430 can effectively detect a level of hemolysis contained in the sample 111. Signals from any additional sensors in the diagnostic sensor assembly 103 may also be received and processed. Test results and other information may be transmitted to the hospital information system (HIS) 101 in some embodiments.

Again referring to FIG. 1, the diagnostic analyzer 100 can include a cartridge receiver 102P, which can be a port, opening, or other suitable coupling feature configured to receive and couple to the diagnostic cartridge 106. Diagnostic cartridge 106 may be coupled or connected to (e.g., inserted in) the cartridge receiver 102P, and by doing so can make an electrical connection between the diagnostic sensor assembly 103 and the controller 105 to allow processing. As shown, the cartridge receiver 102P can comprise a slot sized to receive the diagnostic cartridge 106 therein. Diagnostic cartridge 106 may resemble a playing card being thin as compared to its width and length. For example, the diagnostic cartridge 106 may have a length of about 85 mm, a width of about 55 mm, and a thickness of about 1.2 mm. However, diagnostic cartridge 106 may include other dimensions and/or shapes.

As shown in FIG. 1, diagnostic sensor assembly 103 can be configured as part of the cartridge body 108 of the diagnostic cartridge 106 that is configured for connection with the diagnostic analyzer 100. The diagnostic sensor assembly 103 includes a sample inlet 109, which can be a port, opening, or receiving element, or the like, configured to receive the sample 111 to be tested therein. Sample inlet 109 may be provided on a top layer 108T of the cartridge body 108 as shown in FIG. 1. Sample inlet 109 may comprise a circular or otherwise shaped opening providing a port configured to receive the sample 111 therein. In some embodiments, sample inlet 109 may have a width or diameter dimension of about 4 mm to about 8 mm, although other diameters, dimensions, or shapes may be used.

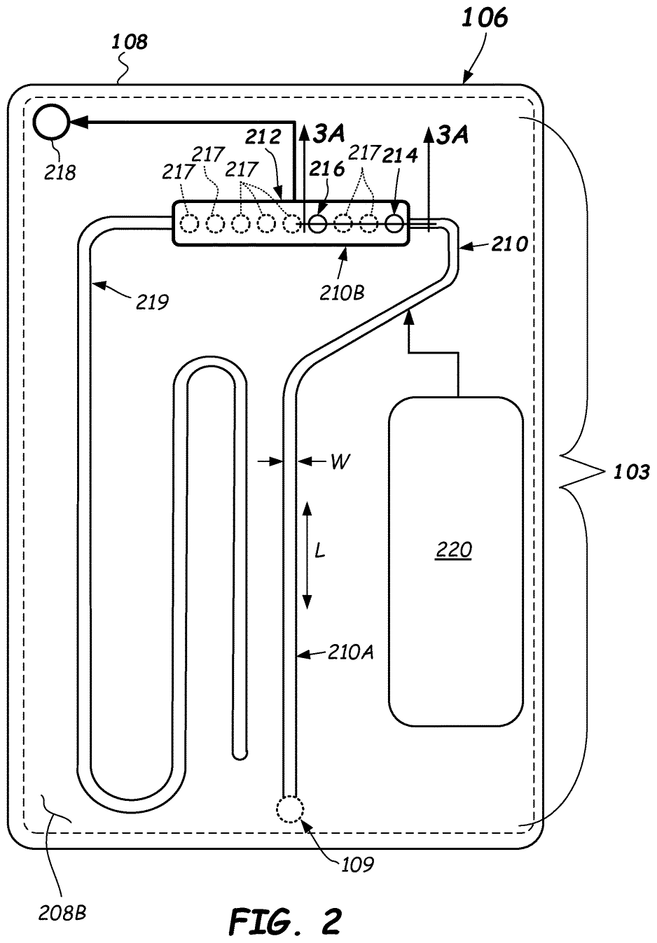

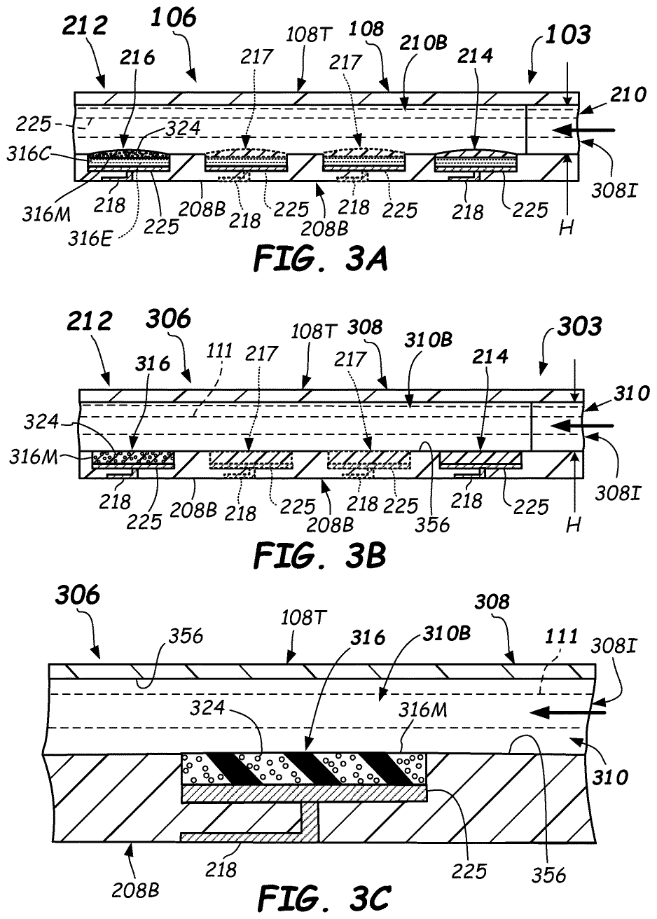

Sample 111 may be whole blood and sample inlet 109 may be configured to allow to a syringe or other suitable pump or transfer device to be sealingly coupled to the sample inlet 109 in order to receive the sample 111 therein and to inject and flow the sample 111 into a sample passageway 210 (shown in FIG. 2). The cartridge body 108 may be made of multiple layers of material adhered together to form the sample passageway 210 therein. For example, the cartridge body 108 may include a bottom layer 208B (FIG. 2), a top layer 108T (FIG. 1), and possibly an intermediate layer 308I (FIGS. 3A-3C and 3E) that may be coupled together and sealed by any suitable means, such as by using an adhesive, a mechanical coupling, a combination thereof, or the like. Portions of the sample passageway 210 may be formed by interaction of mating portions of the top layer 108T, bottom layer 208B, and intermediate layer 308I, if present. In some embodiments, the top layer 108T, bottom layer 208B, and intermediate layer 308I may be formed from the same type of material. For example, the material may be a plastic, such as polypropylene. Alternatively, different materials (e.g., different types of plastic, paper, foil, and/or laminates thereof) may be used for the intermediate layer 108I, top layer 108T, and bottom layer 208B. In some embodiments, the top layer 108T and/or bottom layer 208B may be a clear (transparent or translucent) material so that the flow of the sample 111 therein may be visually observed. Other constructions that form the sample passageway 210 may be used, such as other 2-piece, three-piece, or other multi-piece designs.

Referring again to FIG. 2, a bottom view of the diagnostic cartridge 106 comprising the diagnostic sensor assembly 103 is shown. In the depicted embodiment, the sample passageway 210 can comprise a first portion 210A extending from the sample inlet 109 to a second portion 210B. Second portion 210B can comprise a sensor array 212 therein made up of multiple sensors including at least the two oxygen sensors. The sample passageway 210 can extend from the sample inlet 109 into the cartridge body 108 in some embodiments. The second portion 210B can have different dimensions as compared to the first portion 210A. For example, the second portion 210B may be wider to accommodate the dimensions of the various sensors 214, 216, 217 housed therein. Thus, the second portion 210B may resemble a chamber in some embodiments. Coupled at a downstream end of the second portion 210B can be a waste passageway 219 comprising a conduit or passage that is configured to receive the sample outflow after the sample 111 contacts the last sensor or component in the sensor array 212, such as an additional sensor or a ground 217.

Sample passageway 210 can have a cross-sectional area of from 12,500 um² to 0.8 mm², for example. In some embodiments, the sample passageway 210 can have a width-to-height ratio W:H that may be about 5:1 or greater. Height H is the dimension across the sample passageway 210 as shown in FIG. 3A, whereas the width W is across the sample passageway 210 as shown in FIG. 2. Width W may be from about 250 μm to about 2 mm, and a height H may be from about 50 μm to about 400 μm. The length L along the sample passageway 210 from the sample inlet 109 to the start of the waste passageway 219 may be from about 1.25 mm to about 100 mm or greater. Other relationships between length L, width W, and/or height H may be employed and other suitable length L, height H, and/or width W dimensions may be used.

In more detail, the sensor array 212 of the diagnostic sensor assembly 103 comprises a main oxygen sensor 214 configured to contact the sample 111 along the sample passageway 210 and a modified oxygen sensor 216, 316 also configured to contact the sample 111 along the sample passageway 210. Both of the oxygen sensors 214, 216 or 316 may be provided in the second portion 210B as shown in the depicted embodiments of FIGS. 2-3C and 3E. The modified oxygen sensor 216, 316 may be located downstream (to the left as shown in FIG. 2) of the main oxygen sensor 214 so that the oxidant 324 (FIG. 3A-3C and 3E) associated with the modified oxygen sensor 216, 316 will not change the sample 111 exposed to the main oxygen sensor 214. Other additional sensors and/or a ground 217 may be provided between the main oxygen sensor 214 and the modified oxygen sensor 216, 316, or otherwise located in the sensor array 212.

The modified oxygen sensor 216, 316 comprises the oxidant 324 (FIGS. 3A-3C, and 3E) that is configured to oxidize extracellular (free) hemoglobin to methemoglobin. In particular, the oxidant 324 is configured to oxidize the extracellular hemoglobin iron from a ferrous state (Fe⁺²) to a ferric state (Fe⁺³). In the various depicted embodiments, partial cross-sectioned views are shown in FIGS. 3A-3C and 3E.

In the embodiment of FIG. 3A, the oxidant 324 can be embodied as part of a membrane 316M. The membrane 316M may be formed from any semi-permeable, wettable material, such as a polymer material. For example, the polymer material may be a polyurethane-based material, a polyacrylate-based material, copolymers thereof, or the like. A sensor chamber 316C at least partially formed by the membrane 316M of the modified oxygen sensor 216 can be provided with an electrolyte 316E provided in a sensor chamber 316C.

The electrolyte 316E can be any solid-state proton conducting polymer, such as a mixture of Nafion™ and polyvinylpyrrolidone, which can be mixed in a 4:1 ratio, for example. Optionally, the electrolyte 316E can be a hydrogel comprising poly-N-vinylpyrrolidone K90 (PNPV) and 2, 6 bis (4-azidobenzylidene)-4-methylcyclohexanone, for example. Other suitable liquid or gel electrolytes may be used that are suitable for such pump-type and Clark-type oxygen sensors.

The oxidant 324 can be compounded into the polymer material forming the membrane 316M. The oxidant 324 can be homogeneously included in the membrane 316M, or optionally included in a graded condition, i.e., with a concentration gradient wherein a higher concentration of the oxidant 324 can be provided at the surface of the membrane 316M adjacent to the sample 111 located in the second portion 210B of the sample passageway 210. For example, the gradation can be driven by layers of the same polymer material deposited with various levels of the oxidant 324 being present, with the highest concentration being provided at the outermost layer adjacent to the sample 111.

In principle, membrane 316M, containing the desired membrane base material(s) and including the oxidant 324, can be formed by any suitable process, such as deposition with a volatilizable liquid. In such deposition, a liquid mixture can be dispensed from a tip. Optionally, the membrane 316M may be formed by spin coating, dip coating, screen printing, spray coating, and the like.

In the embodiments of FIGS. 3B and 3C, the oxidant 324 can be compounded into the matrix of a heterogeneous-type of membrane 316M as shown in the modified oxygen sensor 316. In this heterogeneous embodiment of FIGS. 3B and 3C, the membrane 316M is provided in direct contact with the sample 111 and is located between the sample 111 and one or more electrodes 225. For illustration purposes, the one or more electrodes 225 is shown as a single electrode (e.g., a working electrode). The reference electrode may be located at a different location. However, it should be understood that in some embodiments, the one or more electrodes 225 may be comprised of a working electrode and counter electrode, and/or reference electrode. Any conventional electrode construction or arrangement may be used.

The membrane 316M can comprise a hydrophobic polymer admixed with a hydrophilic component. For example, the membrane 316M can comprise such a heterogeneous membrane composition that has a hydrophilic electrolyte-containing compartment and a hydrophobic compartment that supports gas (e.g., O₂) and water vapor transport. The hydrophobic compartment can comprise a polymer.

Example polymer materials for this membrane 316M can include poly-siloxanes, poly-organo-phosphazenes, poly-1 trimethyl-silyl-1-propyne, poly-4-methyl-2-pentyne, and mixtures thereof. The hydrophilic component of the admixture can comprise a hydrophilic polymer such as a polyvinyl alcohol (PVA), a poly-acrylate polymer like a hydroxymethacrylate, a poly-acrylamide, a poly-saccharide, a cellulosic polymer, and/or a gelatin, for example. The hydrophilic component may further include some or all of the following: emulsifier, hydrophilic polymer binder, electrolyte salt, viscosity modifier, and other optional dissolved components. Other optional constituents of the hydrophilic compartment could include one or more components such as a cross-linker, catalyst, redox agent, buffer, and/or surfactant that can be incorporated into the membrane 316M upon formation.

Gas diffusion coefficients of the various phases should differ by 10 or more, 50 or more, or even 100 or more, with the hydrophobic component being significantly higher. In one method, the membrane 316M and oxidant 324 can be deposited in a well formed in the bottom layer 208B, for example. Bottom layer 208B may be an electrically-insulating material, such as an epoxy layer, a polymer composite material, or other suitable electrically insulating material. The membrane-forming solution may be dried down to form the membrane 316M comprising the heterogeneous membrane. Further discussion of the construction and materials of such conventional heterogeneous sensors can be found in U.S. Pat. No. 7,094,330.

In the embodiment of FIG. 3E, the sensor 316 can comprise a membrane 316M containing multiple layers. For example, a base layer 316B may be any oxygen permeable, non-wettable material, such as a polyethylene or polytetrafluoroethylene (PTFE) material, silicone, paraffin wax, or the like. The top layer 316T is configured to be in contact with the sample 111 and may be made of a wettable material, such as for example, a polyurethane-based material, a polyacrylate-based material, copolymers thereof, or the like. The wettable material comprising the top layer 316T contains the oxidant 324 therein.

Wettable material of the membrane 316M of the FIG. 3A embodiment and top layer 316T of the FIG. 3E embodiment are hydrophilic and may comprise a contact angle of greater than 0 degrees and less than or equal to 90 degrees. In some embodiments, the surface of the wettable material can have a contact angle of less than 45 degrees, or even less than 30 degrees. In some embodiments, the surface of the membrane 316M and top layer 316T of the wettable material can be modified to further enhance its wettability and the availability of the oxidant 324.

For example, the top surface of the membrane 316M (FIG. 3A) and top surface of the top layer 316T (FIG. 3E) may be treated in some manner to enhance wettability. For example, the top surface may be plasma treated, i.e., treated with ionized gas and/or radicals, for a sufficient time (e.g., 10 seconds to 5 minutes) to obtain a lower contact angle. Optionally, the top surface may be treated with ultraviolet ozone (UVO) for a suitable time (e.g., about 5 minutes) to obtain a lower contact angle. Other suitable treatment methods for enhancing wettability as well as combinations of the aforementioned contact angle lowering treatments may be used.

Measurements of wettability can be measured by an optical tensiometer and using the sessile drop method. In some embodiments, the oxidant 324 can be provided in a higher concentration in an outer portion of the top layer 316T adjacent to the sample 111 in order to maximize the amount of oxidant 324 that is available to oxidize any extracellular hemoglobin contained in the sample 111.

In all embodiments described herein, the oxidant 324 included in the modified oxygen sensor 316 can comprise potassium ferricyanide, for example. Optionally, the oxidant 324 can comprise any chemical compound from other known classes of oxidants such as organic nitrates or inorganic nitrites, aromatic amines, or quinones, for example that operates to cause oxidation of extracellular hemoglobin to methemoglobin (Fe²⁺ to Fe³⁺) in the sample 111. The oxidant 324 can be provided in an effective amount to cause a sufficient oxidation of extracellular hemoglobin to methemoglobin (Fe²⁺ to Fe³⁺) in the sample 111. The goal is to have sufficient concentration of the oxidant 324 available to oxidize hemoglobin in order to provide a sufficient difference in sensed readings between the main oxygen sensor 214 and the modified oxygen sensor 216, 316. In particular, the oxidant 324 used should not cause interference with the readings of any other sensor or sensors along the sample passageway 210, 310. In some embodiments, such as in the shown in FIGS. 3B-3C, one or more additional thin layers of the membrane 316M may be formed adjacent to the one or more electrodes 225 that can be of the same material as the membrane 316M but that is/are devoid of the oxidant 324 so that any deleterious redox type interactions at the one or more electrodes 225 may be minimized.

In any of the above-described embodiments, the oxidant 324 can be provided in, i.e., compounded into, the membrane 316M (FIGS. 3A-3C and 3E). The oxidant 324 can be provided in an suitable amount, i.e., weight percentage (wt %), of about 0.01 wt % to about 25 wt %, or even from about 0.5 wt % to about 5.0 wt % in some embodiments, based on the total weight of the membrane 316M including the oxidant 324.

As should be understood, the oxidant 324 of the membrane 316M causes oxidation of extracellular hemoglobin to methemoglobin (Fe²⁺ to Fe³⁺), which subsequently releases bound oxygen from the extracellular hemoglobin in the sample 111 in a localized manner. Accordingly, the released/generated oxygen is detectable by the modified oxygen sensor 216, 316 and thus when free (extracellular) hemoglobin is present in the sample 111, quantification of hemolysis of the sample 111 can be obtained in a differential manner (e.g., as difference between the readings from the main oxygen sensor 214 and the modified oxygen sensor 216, 316) as will be further explained herein.

The main oxygen sensor 214 is configured to provide a first measurement correlated to a partial pressure of oxygen in the sample 111 proximate to the main oxygen sensor 214. Similarly, the modified oxygen sensor 216, 316 is configured to provide a second measurement correlated to a partial pressure of oxygen in the sample 111 proximate the modified oxygen sensor 216, 316. In samples 111 that have free hemoglobin therein, excess oxygen will be released by the oxidation reaction between the extracellular hemoglobin in the sample 111 and the oxidant 324, which then can be sensed locally by the modified oxygen sensor 216, 316. A differential measurement as compared with a reading from the main oxygen sensor 214 can then be obtained.

Referring again to FIGS. 3C and 3E, examples of modified oxygen sensors 316 having a solid state integrated chip structures are shown. The modified oxygen sensors 316 may be provided in a diagnostic cartridge 306 as shown in FIG. 3D, which in use may be connected to an inlet 351 and an outlet 352 of a diagnostic analyzer 100 (FIG. 1) or otherwise connected to the sample passageway 210 and waste passageway 219. Modified oxygen sensors 316 of FIGS. 3C and 3E may be included in a diagnostic cartridge like is shown in FIG. 2.

As shown in FIG. 3D, the inlet 351 supplies the sample 111 to the sensor array 212 including the main oxygen sensor 214 and the modified sensor (MO₂) 316. The diagnostic cartridge 306 may further include one or more additional sensors and/or ground 217. The additional sensors may be configured to measure other analytes and/or conditions, such as Cl−, Mg++, Na+, K+, pCO2, Ca++, glucose, lactate, creatinine, and the like. Other additional sensors 217 that are configured to sense other analytes (e.g., BUN, Hct, and/or TCO₂) and/or conditions (e.g., pH) may be included in addition or in substitution thereof.

The diagnostic cartridge 306 may further include one or more reference sensors 354 configured to provide a reference signal. Optionally, the reference signal may be obtained inside of the diagnostic analyzer 100 or elsewhere along the sample passageway 210 or 310. The diagnostic cartridge 306 may further include a common ground at any suitable location that is connectable to the controller 105. The arrangement of the sensors may be other than that shown. However, the modified oxygen sensor 316 may be located downstream of the main oxygen sensor 214. Further, the modified oxygen sensor 316 and the main oxygen sensor 214 may be separated by one or more additional sensors 217 or a suitable space so that the main oxygen sensor 214 is unaffected by the extra oxygen liberated proximate the modified oxygen sensor 316.

The sensors 214, 316, 217 and the one or more reference sensors 354, and the ground (if used) may be electrically coupled to a detection system of the diagnostic analyzer 100 (FIGS. 1 and 4), which may include any suitable electronics to enable reading an electrical potential difference (or current difference) between the main oxygen sensor 214 and the modified oxygen sensor 216, 316 as a measureable signal. Again, the detection system and the reference sensor 354 construction are well known and will not be described further herein. For example, the reference system and the reference sensor 354 can be of the type used in the epoc® blood analysis system available from Siemens Medical Solutions or as is shown in FIG. 3D. However, the reference sensor 354 may be positioned elsewhere along the sample passageway 210, 310 other than on the second portion 210B, 310B.

In more detail and in further reference to FIGS. 3C and 3E, the modified oxygen sensor 316 can comprise a cartridge body 308 including a membrane 316M, which is oxygen permeable, coupled thereto such as to bottom layer 208B. The walls 356 of the diagnostic cartridge 306 and the sensors (e.g., main oxygen sensor 214, modified oxygen sensor 316, reference sensor 354 (see FIGS. 3D, 3E, and 4), and any additional sensors 217) form a second portion 310B that receives the sample 111 therein. The membrane 316M can be formed as a thin polymer sheet that is selective to O₂. The membrane 316M may have a diameter or maximum dimension of from about 200 μm to about 1,700 μm and a thickness of from about 10 μm to 200 μm, for example. Other suitable diameters or maximum dimensions and/or thickness may be used. The chemical composition of the membrane 316M making it selective to O₂ may be as described herein above, or any other suitably oxygen-permeable material.

The modified oxygen sensor 316, reference sensor 354, and any additional sensors and/or ground 317 can comprise one or more electrodes 225, which may be located adjacent to the membrane 316M in some embodiments. The one or more electrodes 225, which may comprise a working, counter, and/or reference electrode, can be made of any suitable construction, such as an electrically conductive trace, masked deposition, or the like. A conductor can extend from each of the one or more electrodes 225, reference sensor 354, and other additional sensor and/or ground 317 to a corresponding electrical contact (e.g., 218 shown in FIGS. 2 and 4), which may be provided on the cartridge body 108, 308 of the diagnostic cartridge 106, 306 (e.g., on a bottom thereof) wherein the electrical contact 218 is interconnectable to the diagnostic analyzer 100 as the diagnostic cartridge 106, 306 is coupled thereto.

For example, the one or more electrodes 225 can comprise a silver (AG) element that can be coated with a silver chloride (AGCl) coating, for example. Optionally, the one or more electrode 225 can comprise gold or platinum, or a combination of any of the aforementioned, for example. Other suitably conductive or combinations of electrically conductive materials can be used. The connection between the one or more electrodes 225 and the electrical contact 218 provided on the cartridge body 108, 308 of the diagnostic cartridge 306 can be any suitable electrically conductive material and may be a trace or conductor formed in any suitable configuration, such as by printing, deposition, or other known conductive conduit or trace-forming methods.

In some embodiments, the sensor arrays 212, 312 may include one or more additional sensors 217 other than the main oxygen sensor 214, and the modified oxygen sensor 216, 316. For example, the other sensors 217 may be configured to sense other analytes and/or conditions as described above. The number of additional sensors 217 can be one or more, five or more, or even 10 or more in some embodiments. The total number of sensors in the sensor array 212, 312 can range from 2-15, for example, including the main oxygen sensor 214, and the modified oxygen sensor 216, 316. As shown in FIGS. 3A, 3B, and 3D, one or more addition sensors 217 may be positioned in the second portion 210B, 310B between the main oxygen sensor 214 and the modified oxygen sensor 216, 316.

In some embodiments, as shown in FIG. 2, the cartridge body 108 of the diagnostic cartridge 106 can comprise a control portion 220 that can be used to supply a calibrator liquid to the sample passageway 210 in order to calibrate the operation of the diagnostic sensor assembly 103 of the diagnostic cartridge 106. Such a control portion 220 is known to persons of skill in the art, and will not be described further herein.

Again referring to FIGS. 1-4, operation of diagnostic analyzer 100 in the depicted embodiments may involve inserting the diagnostic cartridge 106, 306 into cartridge receiver 102P so as to engage the diagnostic sensor assembly 103, 303 of the diagnostic cartridge 106, 306 with the controller 105 via an electrical connection between the diagnostic sensor assembly 103, 303 and controller 105. Sample inlet 109 receives a sample 111 (e.g., contained within a syringe or other conveyance) and the sample 111 is flowed into the sample passageway 210, 310 in order to deliver the sample 111 to the second portion 210B, 310B containing the main oxygen sensor 214 and the modified oxygen sensor 216, 316 and also to any other additional sensor(s) 217 that may be included in the sample passageway 210, 310. Upon contact of the sample 111 with the oxygen sensors 214, 216, 316, signals correlated with oxygen levels sensed by the oxygen sensors 214, 216, 316 are provided to the controller 105. Controller 105 may then perform processing of the various sensor signals received in order to generate a sensed level of the analyte, condition, or component (e.g., O₂) being sensed for each sensor 214, 216, 316, and 217. From the sensed signals from the main oxygen sensor 214 and modified oxygen sensor 216, 316, the controller 105 can operate to detect a level of hemolysis in the sample 111.

Waste sample fluid after passing through second portion 210B, 310B may flow into the waste passageway 219 for storage. Waste passageway 219 may be formed, for example, as a trench, groove, or similar structure within the cartridge body 108 of diagnostic cartridge 106, 306, and may have a serpentine shape or other non-straight shape in some embodiments. Waste passageway 219 should have a length sufficient to hold the waste liquid (e.g., sample 111 after passing through second portion 210B, 310B and possibly control liquid from calibration operation of the control portion 220).

Again referring to FIGS. 3A-3C, the diagnostic cartridge 106, 306 can include one or more electrodes 225 for each sensor 214, 216, 217, 316, and 354 and each is electrically connected to an electrical contact 218. As shown for simplicity in FIG. 2, only one electrical contact 218 is shown, but it should be understood that each of the sensors 214, 216, 217, 316, 354 have a dedicated one or more of the electrical contacts for providing signals, such as an electrical voltage or current. Any potential or current changes from the operation of the sensors can then be detected. If the sensor type is designed to be potentiometric, then a baseline voltage may be provided, and changes in that baseline voltage may be detected. For example, a low voltage of from 1 mV to 500 mV, or even from 1 mV to 50 mV, may be provided as the baseline voltage in the measurement circuit. Likewise, if the sensor measurement system is amperometric, then a baseline electrical current may be provided from which changes can be detected. For example, a low current of from 1 nA to 500 nA, or even 1 nA to 50 nA, may be provided as the baseline current in the measurement circuit. Other suitable voltage or current baseline values can be used. Any conventional circuit enabling the measurement of voltage and/or current may be used.

As shown in FIG. 4, for example, each of the one or more electrodes of the main oxygen sensor 214, modified oxygen sensor 216, 316, additional sensor or ground 217, and reference sensor 354 may have a conductive path extending to the controller 105. For example, in some embodiments there may be conductive paths coming from and going to corresponding electrical contacts 218 configured on the surface of the cartridge body 108, 308 of the diagnostic cartridge 106, 306. For example, as shown in FIG. 4, conductive paths may include connections to each of electrical contacts 218A, 218B (only a few labeled). The electrical contacts 218 are in turn contacted by engaging contacts 221 (e.g., engaging contacts 221A, 221B—only a few labeled) of the electrical connector 458 to connect to the controller 105. Likewise, the ground 217 (if used) may be connected to contact 218G, which is contacted by engaging contact 221G of the electrical connector 458. These signals from the main oxygen sensor 214 and the modified oxygen sensor 216, 316 and any optional additional sensors 217 and reference sensor 354 can be effectively received by the controller 105. The electrical connection between the electrical contacts 218 and the engaging contacts 221 may be made upon coupling the diagnostic cartridge 106, 306 to the cartridge receiver 102P.

Again referring to FIG. 4, the diagnostic analyzer 100 may also include a hemolysis detection module 430 in the controller 105. The hemolysis detection module 430 may be stored as programmed code in the memory 431 and executed by processor 432. Processor 432 may control operation of sensors 214, 216, 316, 354, 217, memory 431, and/or display 104D. The signals from the various sensors 214, 216, 217 may be manipulated in order to provide values that are correlated to the analyte being sensed. In the case of the main oxygen sensor 214 and modified oxygen sensor 216,316, the signals therefrom are manipulated to derive values correlated to oxygen levels thereof. From these signal values, the hemolysis detection module 430 may determine, based upon a difference there between, a level of hemolysis in the sample 111. The difference can be calculated by way of a difference-finding program code. The larger the difference found via the difference-finding program of the hemolysis detection module 430 is indicative of more oxygen freed as a result of the oxidation reaction with oxidant 324 occurring proximate the modified oxygen sensor 216, 316, such difference being correlated with the level of hemolysis (extracellular hemoglobin) present in the sample 111. The larger the difference determined or calculated via the difference-finding program, the higher the level or amount of hemolysis present in the sample 111.

Processor 432 may be any suitable computational resource such as, but not limited to, a microprocessor, a microcontroller, an embedded microcontroller, a digital signal processor (DSP), a field programmable gate array (FPGA) that is configured to perform as a microcontroller, or the like.

Memory 431 may be any suitable type of memory, such as, but not limited to, one or more of a volatile memory, a non-volatile memory, or combinations thereof. Volatile memory may include, but is not limited to, a static random access memory (SRAM), or a dynamic random access memory (DRAM). Non-volatile memory may include, but is not limited to, an electrically programmable read-only memory (EPROM), an electrically erasable programmable read-only memory (EEPROM), a flash memory, etc. Memory 431 may have a plurality of instructions stored therein that, when executed by the processor 432, cause the processor 432 to perform various actions specified by one or more of the stored plurality of instructions, including the difference-finding program.

User interface may include one or more display screens (e.g., display 104D). The user interface may be controlled by processor 432, and functionality of the user interface may be implemented, at least in part, by computer-executable instructions (e.g., program code or software) stored in memory 431 and/or executed by processor 432 of the diagnostic analyzer 100. In some embodiments, processor 432 may receive measured results from the oxygen sensors 214, 216, 316 and also from additional sensor(s) 217 if other additional sensor or sensors like reference sensor 354 are included, process the measured results to generate calculated results, and present the calculated results and/or other information, such as patient information, via display 104D of the user interface. For example, the user interface and display 104D may be configured to present one or more measured and/or calculated results of hemolysis and possibly other analyte and/or condition measurements to a user of the diagnostic analyzer 100. Such results may be communicated to the HIS 101, as well.

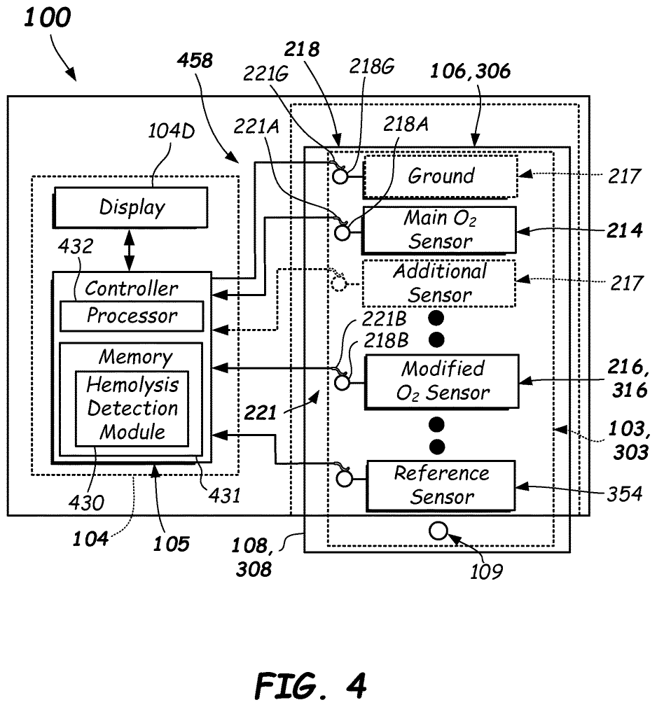

FIG. 5 illustrates an alternate embodiment of a diagnostic sensor assembly 503 that can be included in a diagnostic cartridge 506. This embodiment includes a sample passageway 510 similar to previous embodiments, but the sample passageway 510 includes at least two forks or branches, such as first passageway 510B and second passageway 510C. The first passageway 510B and the second passageway 510C can optionally split off from a primary passageway 510A that extends from the sample inlet 109 in some embodiments. In this embodiment, a main oxygen sensor 214 can be located on the first passageway 510B, and the modified oxygen sensor 516 can be provided on the second passageway 510C. For example, the main oxygen sensor 214 can be provided in the first passageway 510B split from a primary passageway 510A and the modified oxygen sensor 516 can be provided in the second passageway 510C split from the primary passageway 510A. Optional one or more additional sensors or ground 217 may be provided on one or both of the first passageway 510B and the second passageway 510C of the same type discussed above, for example. Other sensor types may be included alternatively, or in addition. Likewise, in each of the FIG. 5 embodiments described herein, a reference sensor may be included anywhere along one or both of the first and second passageways 510B, 510C or as part of the oxygen sensors 214, 214B. Again, for ease of illustration electrical contacts 518 are shown as a single circle, but each of the main oxygen sensor 214, oxygen sensor 214B, additional sensor and/or ground 217, and reference sensor 354 may have its own electrical contact. The diagnostic cartridge 506 including the diagnostic sensor assembly 503 may connect with the diagnostic analyzer 100 in the same manner as is shown in FIG. 4.

Because sample passageway 510 is bifurcated and the two oxygen sensors 214, 516 are not located in series within the same, single sample passageway, there is advantageously reduced or no risk that the oxidant 524 associated with the modified oxygen sensor 516 will change the sample exposed to the main oxygen sensor 214. Like before, the diagnostic cartridge 506 can include a control portion 220 as well as waste passageways 219 extending from each of the first passageway 510B and the second passageway 510C.

This embodiment of the modified oxygen sensor 516 differs in that the oxidant 524 of the modified oxygen sensor 516 is provided at a location in the second passageway 510C enabling the sample 111 to flow over the oxidant 524 and oxidize extracellular hemoglobin from the ferrous state (Fe⁺²) to the ferric state (Fe⁺³), which then liberates oxygen that can be sensed by the oxygen sensor 214B. Oxygen sensor 214B can be identical to the main oxygen sensor 214 located in the first passageway 510B, and can be of conventional construction. As shown, the modified oxygen sensor 516 is made up of or comprises the oxidant 524 and the oxygen sensor 214B, wherein the oxygen sensor 214B can be conventional like main oxygen sensor 214. The oxidant 524 may be located and positioned upstream of the oxygen sensor 214B.

In an alternative embodiment, the modified oxygen sensor 516 may differ from the main oxygen sensor 214 in the same manner as described above with respect to modified oxygen sensor 216, 316 where the oxidant 324 is contained in its membrane 316M. This alternative embodiment differs from the embodiments of FIG. 2 in that the modified oxygen sensor 516 can be located in a different branch of the sample passageway 510 (e.g., second passageway 510C) and thus the first measurement and the second measurement can be obtained in any order. For example, in this alternative embodiment of FIG. 5, the first and second measurements may be obtained simultaneously (or any other order) depending on location of the oxygen sensors in their respective passageway. The use of the bifurcated passageway can operate to reduce or minimize the risk of modified oxygen sensor 516 changing the sample exposed to the main oxygen sensor 214, and the removal of the requirement that the sample must flow past the main sensor prior to flowing past the modified sensor.

In another embodiment, the oxidant 524 may be provided upstream of the oxygen sensor 214B in the second passageway 510C and also in the membrane of the oxygen sensor 214B. The amounts in each location may be adjusted to achieve maximum oxidization hemoglobin to methemoglobin (Fe²⁺ to Fe³⁺).

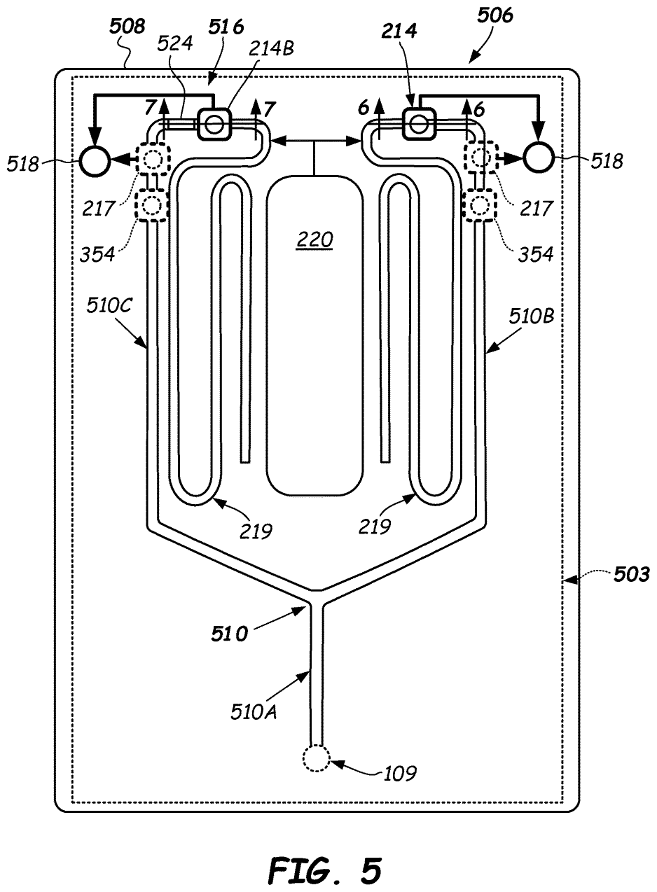

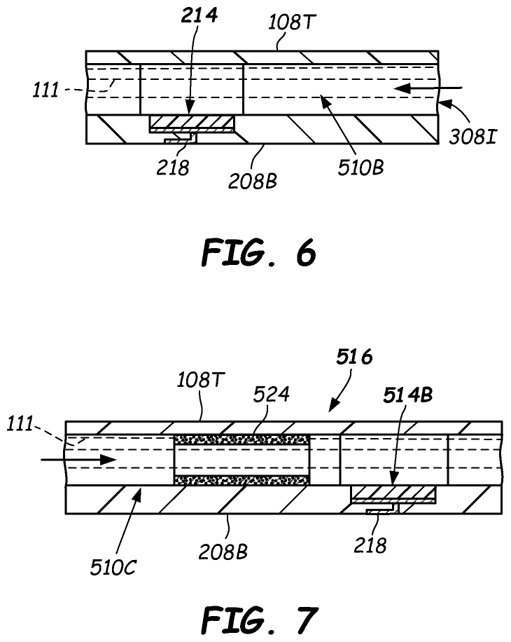

FIGS. 6 and 7 illustrate partial cross-sectional views taken along section lines 6-6 and 7-7 of FIG. 5, respectively. The diagnostic cartridge 506 can be made up of multiple layers adhered together as before. Each of the first passageway 510B and second passageway 510C can include an oxygen sensor 214, 214B, which can both be conventional oxygen sensors, and the same as main oxygen sensor 214 previously described. The second passageway 510C can include the oxidant 524. The oxidant 524 can be located at a position upstream from the oxygen sensor 514B. The oxidant 524 can be provided in any suitable form, such as in the form of a split cylinder or other shaped member that can be sprayed on and dried down on passageway portions of one or both of the top layer 108T and bottom layer 208B, or even on intermediate layer 308I thereof.

In this embodiment, the oxidant 524 can be provided with or without a binder material, such as a dissolvable aqueous polymer material such as polyvinyl alcohol, polyacrylic acid, or the like. The oxidant 524 may be mixed with the dissolvable aqueous polymer material. Dissolvable aqueous polymer material may be dissolvable by contact with the sample 111. The binder material may optionally be a non-dissolvable material (e.g. cross-linked) but sufficiently wetable or porous to allow sufficient interaction with the sample 111 to facilitate oxidation of the extracellular hemoglobin present. With the binder being not dissolvable, this may allow for repeat use conditions versus single use applications. In this configuration, the oxidant 524 may also be potassium ferricyanide. However, other suitable oxidant materials that can sufficiently oxidize hemoglobin may be used.

The oxidant 524 may be provided in about 0.01-99 wt % based on the total weight of the binder and the oxidant 524. It may be desirable to have a very low amount of the binder in relation to the oxidant 524. For example, some binders can be used in about 1 wt % to about 3 wt % as a means to just “hold” the oxidant 524 in place until dissolved by the sample 111. The thickness and length of the cylinder or other shaped member including the oxidant 524 can be of a sufficient length and thickness or dimension to enable a suitable measurable change in oxygen level proximate the oxygen sensor 214B when free extracellular hemoglobin is present as compared to the oxygen level at the main oxygen sensor 214.

In some embodiments, the oxidant 524 may be provided in one of the wells in the bottom layer 208B located upstream from the oxygen sensor 214B, like a well containing the membrane 316M in modified oxygen sensor 316 of FIG. 3B. However, in this embodiment, the well need not include an electrode 225. The oxidant 524 may be compounded in a membrane formed in the well or provided in some other form, such as a form dissolvable by the sample as described above.

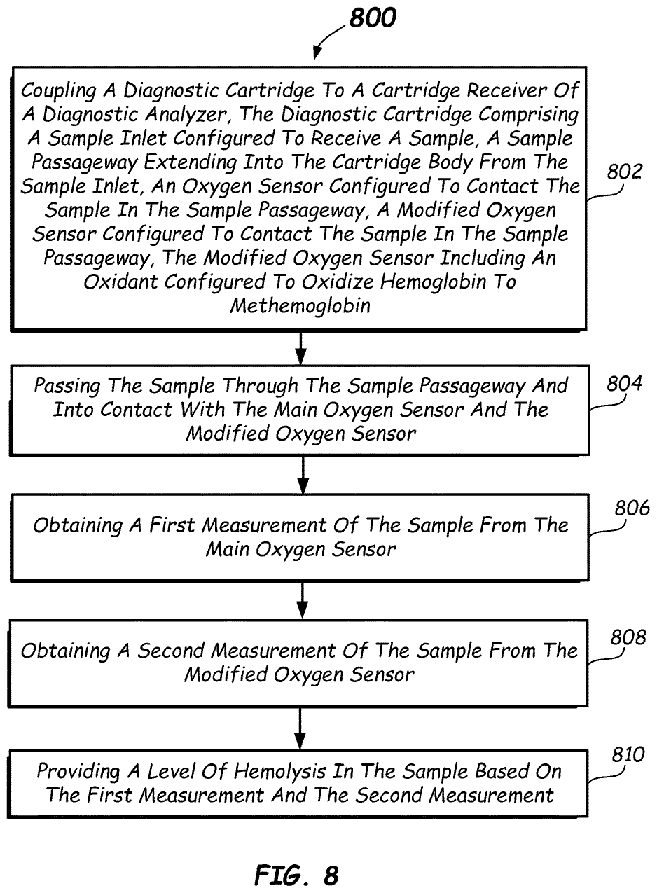

FIG. 8 illustrates a flowchart of an example of a method 800 of determining hemolysis of a sample 111 using the diagnostic sensor assembly 103, 303, 503 and the diagnostic analyzer 100 in accordance with embodiments of the present disclosure. Method 800 begins with block 802 by coupling the diagnostic cartridge 106, 306, 506 to the cartridge receiver 102P of the diagnostic analyzer 100. For example, diagnostic cartridge 106, 306, 506 may be coupled to (e.g., inserted into) cartridge receiver 102P of the analyzer body 102, which makes electrical connection between the diagnostic sensor assembly 103, 303, 503 of the diagnostic cartridge 106, 306, 506 and the controller 105.

In the depicted embodiment, the diagnostic sensor assembly 103, 303, 503 of the diagnostic cartridge 106, 306, 506 comprises a sample inlet 109 configured to receive a sample 111, a sample passageway 210, 310, 510 extending into the cartridge body 108, 308, 508 from the sample inlet 109, a main oxygen sensor 214 configured to contact the sample 111 in the sample passageway 210, 310, 510, a modified oxygen sensor 216, 316, 516 configured to contact the sample 111 in the sample passageway 210, 310, 510, the modified oxygen sensor 216, 316, 516 including an oxidant 224, 324, 524 configured to oxidize extracellular hemoglobin iron to methemoglobin as stated above. In some embodiments, prior to use of the diagnostic analyzer 100, display 104D and/or user interface may prompt a user to couple the diagnostic cartridge 106, 306, 506, enter user identification, scan a name tag or other barcode, enter a password or otherwise authenticate their identity, and/or to provide the sample 111 into the sample inlet 109.

Once the cartridge body 108, 308, 508 has been coupled to the cartridge receiver 102P of the diagnostic analyzer 100, in block 804, the method 800 may further comprise passing the sample 111 through the sample passageway 210, 310, 510 and into contact with the main oxygen sensor 214 and the modified oxygen sensor 216, 316, 516. The sample 111 will also contact any additional sensors or ground 217, and reference sensor 534 that are positioned along the sample passageway 210, 310, 510. For example, a syringe or other sample delivery device may be employed to interface with sample inlet 109 of cartridge body 108, 308, 508. The syringe or other device may move sample 111 into the sample inlet 109 through the sample passageway 210, 310 510 and into the waste passageway 219.

The method 800 further comprises, in block 806, obtaining a first measurement of the sample 111 from the main oxygen sensor 214, and, in block 808, obtaining a second measurement of the sample 111 from the modified oxygen sensor 216, 316, 516. In block 810, the method 800 comprises providing a level of hemolysis in the sample based on the first measurement and the second measurement. Such information may be collected by controller 105 and the level of hemolysis may be displayed on user interface (e.g., on display 104D) for communication to a user of the diagnostic analyzer 100. Additionally, any other test results and other information as well as the level of hemolysis may be transmitted to the hospital information system (HIS) 101 either though a hardwired connection or wirelessly. For example, a WIFI or hardwired LAN communication can be used.

Sample 111 used for the testing may amount to 100 μL or less, or even than 50 μL or less in some embodiments, although other sample 111 amounts may be used. As should now be apparent, the diagnostic sensor assembly 103, 303, 503 embodied in a diagnostic cartridge 106, 306, 506 may be used in a diagnostic analyzer 100 for performing multiple oxygen measurements in order to detect a level of hemolysis in the sample 111. As should be also recognized, the diagnostic sensor assembly 103, 303, 503 may be included in a diagnostic analyzer without being embodied in a diagnostic cartridge and may include a wash system interfacing therewith in order to reuse the diagnostic sensor assembly 103, 303, 503.

In some embodiments, only the sensor array 212, 312 may be included as a disposable cartridge that can couple to the diagnostic analyzer and the diagnostic analyzer itself can include the sample inlet and passageway, waste passageway, control portion, and a wash system enabling washing of the sensor array 212, 312 after use. Thus, the diagnostic cartridge can include an inlet (like inlet 351) and an outlet (like outlet 352) that sealingly connect to the passageway and the waste passageway of the diagnostic analyzer upon coupling the cartridge to the analyzer. Thus, in this instance, the diagnostic cartridge includes the main and modified oxygen sensors as described herein as well as the electrical contacts so that the cartridge is removable/detachable from the diagnostic analyzer after multiple uses.

The following is a list of non-limiting illustrative embodiments disclosed herein:
1. A diagnostic sensor assembly, comprising:
   a sample inlet configured to receive a sample;
   a sample passageway extending from the sample inlet;
   a main oxygen sensor configured to contact the sample along the sample passageway; and
   a modified oxygen sensor configured to contact the sample along the sample passageway, the modified oxygen sensor comprising an oxidant configured to oxidize hemoglobin to methemoglobin, from which quantification of hemolysis of the sample can be obtained.
2. The diagnostic sensor assembly of illustrative embodiment 1, wherein the diagnostic sensor assembly is configured as part of a cartridge body of a diagnostic cartridge that is configured for connection with a diagnostic analyzer.
3. The diagnostic sensor assembly of any one of the preceding illustrative embodiments, wherein the main oxygen sensor is configured to provide a first measurement correlated to a partial pressure of oxygen in the sample.
4. The diagnostic sensor assembly of any one of the preceding illustrative embodiments, wherein the oxidant of the modified oxygen sensor comprises potassium ferricyanide.
5. The diagnostic sensor assembly of any one of the preceding illustrative embodiments, wherein the oxidant is configured to oxidize the hemoglobin from a ferrous state (Fe⁺²) to a ferric state (Fe⁺³).
6. The diagnostic sensor assembly of any one of the preceding illustrative embodiments, wherein the modified oxygen sensor is configured to provide a second measurement correlated to a partial pressure of oxygen in the sample.
7. The diagnostic sensor assembly of any one of the preceding illustrative embodiments, wherein the oxidant is provided in a membrane of the modified oxygen sensor.
8. The diagnostic sensor assembly of any one of the preceding illustrative embodiments, wherein the oxidant of the modified oxygen sensor is provided at a location in the sample passageway enabling the sample to flow over the oxidant and oxidize extracellular hemoglobin from a ferrous state (Fe⁺²) to a ferric state (Fe⁺³).
9. The diagnostic sensor assembly of any one of the preceding illustrative embodiments, wherein the main oxygen sensor is provided in a first passage split from a primary passage and the modified oxygen sensor is provided in a second passage split from the primary passage.
10. A diagnostic analyzer, comprising:
   an analyzer body including a cartridge receiver and an electrical connector;
   a diagnostic cartridge receivable in the cartridge receiver, the diagnostic cartridge comprising:
      a cartridge body configured to be coupled to the cartridge receiver, the cartridge body including:
      an electrical contact couplable to the electrical connector when the cartridge body is received into the cartridge receiver,
      a sample inlet configured to receive a sample;
      a sample passageway extending into the cartridge body from the sample inlet and configured to receive the sample therein;
      a main oxygen sensor configured to contact the sample in the sample passageway and configured to provide a first measurement of the sample;
      a modified oxygen sensor configured to contact the sample in the sample passageway, the modified oxygen sensor including an oxidant configured to oxidize hemoglobin to methemoglobin and configured to provide a second measurement of the sample; and a controller coupled to the electrical connector,
   the controller configured to receive the first measurement and the second measurement of the sample and configured to provide a level of hemolysis in the sample based on the first measurement and the second measurement.
11. The diagnostic analyzer of illustrative embodiment 10, wherein the first measurement is correlated to a first partial pressure measurement of oxygen in the sample.
12. The diagnostic analyzer of any one of the preceding illustrative embodiments, wherein the second measurement is correlated to a second partial pressure measurement of oxygen in the sample.
13. The diagnostic analyzer of any one of the preceding illustrative embodiments, wherein an elevated second measurement as compared to the first measurement is quantitatively correlated to the level of hemolysis in the sample.
14. The diagnostic analyzer of any one of the preceding illustrative embodiments, wherein the controller is configured to determine a difference between the first measurement and the second measurement.
15. The diagnostic analyzer of any one of the preceding illustrative embodiments, wherein the difference is quantitatively correlated to the level of hemolysis in the sample.
16. A method of determining hemolysis of a sample, comprising:
   coupling a diagnostic cartridge to a cartridge receiver of a diagnostic analyzer, the diagnostic cartridge comprising a sample inlet configured to receive the sample, a sample passageway extending into the cartridge body from the sample inlet, a main oxygen sensor configured to contact the sample in the sample passageway, and a modified oxygen sensor configured to contact the sample in the sample passageway, the modified oxygen sensor including an oxidant configured to oxidize extracellular hemoglobin to methemoglobin;
   passing the sample through the sample passageway and into contact with the main oxygen sensor and the modified oxygen sensor;
   obtaining a first measurement of the sample from the main oxygen sensor;
   obtaining a second measurement of the sample from the modified oxygen sensor; and
   providing a level of hemolysis in the sample based on the first measurement and the second measurement.

The foregoing description discloses only example embodiments of the disclosure. Modifications of the above disclosed diagnostic cartridge and diagnostic analyzer and methods of detecting hemolysis that fall within the scope of the invention will be readily apparent to those of ordinary skill in the art. Accordingly, it should be understood that other embodiments may fall within the scope of the invention, as defined by the following claims and their equivalents.

What is claimed is:

1. A diagnostic sensor assembly, comprising:
a sample inlet configured to receive a sample;
a sample passageway extending from the sample inlet;
a main oxygen sensor configured to contact the sample along the sample passageway; and
a modified oxygen sensor configured to contact the sample along the sample passageway, the modified oxygen sensor comprising an oxidant configured to oxidize hemoglobin to methemoglobin, from which quantification of hemolysis of the sample can be obtained.

2. The diagnostic sensor assembly of claim 1, wherein the diagnostic sensor assembly is configured as part of a cartridge body of a diagnostic cartridge that is configured for connection with a diagnostic analyzer.

3. The diagnostic sensor assembly of claim 1, wherein the main oxygen sensor is configured to provide a first measurement correlated to a partial pressure of oxygen in the sample.

4. The diagnostic sensor assembly of claim 1, wherein the oxidant of the modified oxygen sensor comprises potassium ferricyanide.

5. The diagnostic sensor assembly of claim 1, wherein the oxidant is configured to oxidize the hemoglobin from a ferrous state (Fe⁺²) to a ferric state (Fe⁺³).

6. The diagnostic sensor assembly of claim 1, wherein the modified oxygen sensor is configured to provide a second measurement correlated to a partial pressure of oxygen in the sample.

7. The diagnostic sensor assembly of claim 1, wherein the oxidant is provided in a membrane of the modified oxygen sensor.

8. The diagnostic sensor assembly of claim 1, wherein the oxidant of the modified oxygen sensor is provided at a location in the sample passageway enabling the sample to flow over the oxidant and oxidize extracellular hemoglobin from a ferrous state (Fe⁺²) to a ferric state (Fe⁺³).

9. The diagnostic sensor assembly of claim 8, wherein the main oxygen sensor is provided in a first passage split from a primary passage and the modified oxygen sensor is provided in a second passage split from the primary passage.

10. A diagnostic analyzer, comprising:
an analyzer body including a cartridge receiver and an electrical connector;
a diagnostic cartridge receivable in the cartridge receiver, the diagnostic cartridge comprising:
a cartridge body configured to be coupled to the cartridge receiver, the cartridge body including:
electrical contacts couplable to the electrical connector when the cartridge body is received into the cartridge receiver,
a sample inlet configured to receive a sample;
a sample passageway extending into the cartridge body from the sample inlet and configured to receive the sample therein;
a main oxygen sensor configured to contact the sample in the sample passageway and configured to provide a first measurement of the sample;
a modified oxygen sensor configured to contact the sample in the sample passageway, the modified oxygen sensor including an oxidant configured to oxidize hemoglobin to methemoglobin and configured to provide a second measurement of the sample; and
a controller coupled to the electrical connector, the controller configured to receive the first measurement and the second measurement of the sample and configured to provide a level of hemolysis in the sample based on the first measurement and the second measurement.

11. The diagnostic analyzer of claim 10, wherein the first measurement is correlated to a first partial pressure measurement of oxygen in the sample.

12. The diagnostic analyzer of claim 10, wherein the second measurement is correlated to a second partial pressure measurement of oxygen in the sample.

13. The diagnostic analyzer of claim 12, wherein an elevated second measurement as compared to the first measurement is quantitatively correlated to the level of hemolysis in the sample.

14. The diagnostic analyzer of claim 10, wherein the controller is configured to determine a difference between the first measurement and the second measurement.

15. The diagnostic analyzer of claim 14, wherein the difference is quantitatively correlated to the level of hemolysis in the sample.

16. A method of determining hemolysis of a sample, comprising:
coupling a diagnostic cartridge to a cartridge receiver of a diagnostic analyzer, the diagnostic cartridge comprising a sample inlet configured to receive the sample, a sample passageway extending into a cartridge body from the sample inlet, a main oxygen sensor configured to contact the sample in the sample passageway, and a modified oxygen sensor configured to contact the sample in the sample passageway, the modified oxygen sensor including an oxidant configured to oxidize extracellular hemoglobin to methemoglobin;
passing the sample through the sample passageway and into contact with the main oxygen sensor and the modified oxygen sensor;
obtaining a first measurement of the sample from the main oxygen sensor;
obtaining a second measurement of the sample from the modified oxygen sensor; and
providing a level of hemolysis in the sample based on the first measurement and the second measurement.
